(19) United States
(12) Patent Application Publication
(10) Pub. No.: US 2006/0089608 A1
(43) Pub. Date: Apr. 27, 2006

(54) Embryo transfer using tranvaginal ultrasound transducer

(75) Inventors: Marwan M. Shaykh, Jacksonville, FL (US); Rodney W. Bosley, Chester Springs, PA (US); Troy W. Wingler, Bloomington, IN (US)

Correspondence Address: BRINKS HOFER GILSON & LIONE/CHICAGO/COOK, PO BOX 10395, CHICAGO, IL, 60610, US

(73) Assignee: Vance Products Incorporated, Spencer, IL (US)

(21) Appl. No.: 11/229,933

(22) Filed: Sep. 19, 2005

Related U.S. Application Data
(63) Continuation-in-part of application No. 10/264,651, filed on Oct. 4, 2002, now abandoned.
Application No. 10/264,651 is a continuation-in-part of application No. 09/669,315, filed on Sep. 25, 2000, now Pat. No. 6,527,752.
(60) Provisional application No. 60/156,049, filed on Sep. 24, 1999.

(51) Int. Cl. A61M 25/00

(52) U.S. Cl. 604/264

(57) ABSTRACT

An embryo transfer catheter includes an echogenic tip or radiopaque tip and a unique stop that allows a physician to more accurately place embryos in a patient's uterus. The catheter is preferably used with an ultrasound transducer placed in the patient's vagina, allowing the physician to better visualize the distance between the cervical opening and the desired location for placement in the uterus. Abdominal placement of a transducer is also helpful.

### RELATED APPLICATIONS

This application is a continuation-in-part of U.S. patent application Ser. No. 10/264,651, filed Oct. 4, 2002, presently pending, which is a continuation-in-part of U.S. patent application Ser. No. 09/669,315, now U.S. Pat. No. 6,527,752, and also claims the benefit of the filing date under 35 U.S.C. § 119(e), of U.S. Provisional Application 60/156,049, filed Sep. 24, 1999, which is hereby incorporated by reference in its entirety. Each of these applications and patents is hereby incorporated by reference in its entirety, as though each document were reproduced in the text below.

### FIELD OF THE INVENTION

The technical field of the invention is that of assisted reproductive technology, involving human in vitro fertilization (IVF) and embryo transfer (ET).

### BACKGROUND

Human In Vitro Fertilization (IVF) and Embryo Transfer (ET), first successfully performed in 1978, has become a widely practiced procedure to treat infertile couples who have failed with more conventional methods of therapy such as superovulation and intrauterine insemination. The most common indications for IVF and related procedures, such as Gamete In Vitro Fertilization or Gamete Intra-Fallopian Transfer (GIFT) which includes women having blocked or damaged fallopian tubes, and includes low sperm and/or egg quality. Related factors include age of the female, and the degree of endometrial receptivity. The procedure may also be used in cases of severe male factor where direct (intracytoplasmic) injection of sperm is an option.

The IVF/ET procedure typically involves the hormonal stimulation of the female to first suppress her ability to ovulate on her own, then stimulate development of follicles in the ovaries with a fertility medication. The mature eggs are removed from the ovary transvaginally using a needle, preferably guided under ultrasound. Following harvesting of the eggs, the eggs are identified and sorted with regard to maturity, and then placed with a sperm sample from the male. Approximately 24 hours after fertilization, the eggs are examined to confirm fertilization, which occurs in approximately 65% to 85% of the eggs harvested.

After a short development period, the embryos are transferred, along with a volume of fluid, to the uterus using a delivery catheter. The delivery catheter is made of a soft plastic material to avoid damage to the endometrium. There are many potential difficulties in achieving a successful implantation. Because of the soft nature of the standard delivery catheter, in a number of cases, the tip of the catheter may bend back on itself or curve away from the fundus of the uterus. The tip may also accidentally pass between the layer of the endometrium and myometrium. Conversely, a stiffer catheter increases the risk of trauma to the uterus or cervix, with the latter possibly leading to the release of prostaglandins and expulsion of the eggs from the endometrium.

One particular difficulty in achieving successful implantation is the difficulty the surgeon has in visualizing the uterus and the endometrium into which the embryo is implanted. The embryo is desirably transferred onto the rich lining of the uterus (endometrium) without disturbing the lining or causing trauma. As noted above, transfer catheters are typically made from very soft material to minimize trauma. However, the endometrium is very fragile and can be easily disturbed. In addition, transfer catheters may be marked with an echogenic or radiopaque band or feature on the distal tip. This feature allows the physician to visualize the tip when used with an ultrasound probe on the patient's abdomen. However, most surgeons have difficulty visualizing both the uterus and the tip of the catheter using ultrasound with this technique.

Several unsuccessful attempts have been made to improve success rates. U.S. Pat. No. 6,165,165 uses a guiding catheter and an implant catheter, the implant catheter made from materials of two different durometers, so that the stiffness of the catheter decreases from the proximal end to the distal end of the catheter. The resulting catheter may be easier to guide, but is still subject to interference from mucus. WIPO International Patent Applications WO99/37348 and WO01/74417 attempt to solve the problem with an end cap on a guiding catheter that swings open to allow the transfer catheter to pass through an opening and transfer the embryos. Alternatively, the transfer catheter may have a side port rather than an axial port on the distal end, so the side port will avoid interference from mucus. These embodiments are still subject to interference from mucus.

One way to increase the likelihood of success is to tailor the catheters used to the person undergoing the treatment, i.e., by using different lengths of catheter. These attempts to tailor the catheters have led to a proliferation of lengths of catheters, especially in guide catheters. Even with overnight delivery of the desired resources, this results in the need for hospitals and clinics to inventory more catheters and more sets of catheters than is desirable. What is needed is a catheter system and a better technique that can increase the likelihood of successful embryo implantation patients desiring this procedure by better placement of the embryos that are transferred.

### BRIEF SUMMARY

The foregoing problems are solved and a technical advance is achieved in an illustrative transfer or delivery catheter which includes ultrasonically reflective components or features to enhance its visibility under transabdominal or transvaginal ultrasound guidance, during embryo transfer, for example. The present invention helps to increase the likelihood of successful implantation by using a cervical stop to limit the penetration of a transfer catheter into a woman. One aspect of the invention is a transfer system for cellular material, the transfer system comprising a distal end detectably different from adjacent portions of the catheter. The transfer catheter includes a guide catheter for holding and guiding the transfer catheter, the guide catheter further comprising a movable cervical stop. The transfer system also includes a locking mechanism for fixing the position of the transfer catheter with respect to the guide catheter, wherein the transfer system is suitable for guidance using a transvaginal ultrasound technique

Another aspect of the invention is a catheter transfer system. The transfer catheter system includes a soft inner transfer catheter comprising a connector for fluid transfer on a proximal end and an echogenic or radiopaque marker on a distal end. The catheter transfer system also includes an outer guide catheter comprising a cervical stop and a series of spaced marks on a distal end of the outer guide catheter, and a locking mechanism for fixing a position of the inner and outer catheters with respect to one another.

Another aspect of the invention is a method of transferring an embryo. The method includes steps of adjusting a cervical stop on a catheter transfer system, and placing the catheter transfer system near an opening of a cervix. The method also includes observing at least one echogenic or radiopaque feature of the catheter transfer system using an ultrasound transducer placed near a vagina, and also optionally using at least one echogenic or radiopaque feature of the catheter transfer system using an ultrasound transducer placed near an abdomen. The method concludes with steps of advancing a transfer catheter to a desired position in a uterus and transferring the embryo into the uterus.

Another aspect of the invention is a method of transferring cellular material. The method comprises placing at least one catheter near an opening of a cervix, and observing a position of the at least one catheter using an ultrasound transducer placed in or near a vagina, and optionally using an ultrasound transducer placed near an abdomen. The method includes steps of adjusting a cervical stop on the at least one catheter, and advancing the at least one catheter into the cervix and uterus until the stop is reached. The method concludes with steps of observing the position of a distal end of the at least one catheter using at least one ultrasound transducer and transferring the cellular material.

There are many aspects and embodiments of the invention, some of which are described below in a specification and drawings which are meant to be illustrative and descriptive, rather than limiting.

### BRIEF DESCRIPTION OF THE DRAWINGS

FIG. 1-3 depict a first embodiment of a catheter transfer system according to the present invention;

FIGS. 4-5 depict a second embodiment of a transfer catheter system;

FIGS. 6-7 depict a third embodiment of a transfer catheter system; and

FIGS. 8-9 are flowcharts illustrating methods of practicing an improved method of embryo transfer.

### DETAILED DESCRIPTION OF THE DRAWINGS AND THE PRESENTLY PREFERRED EMBODIMENTS

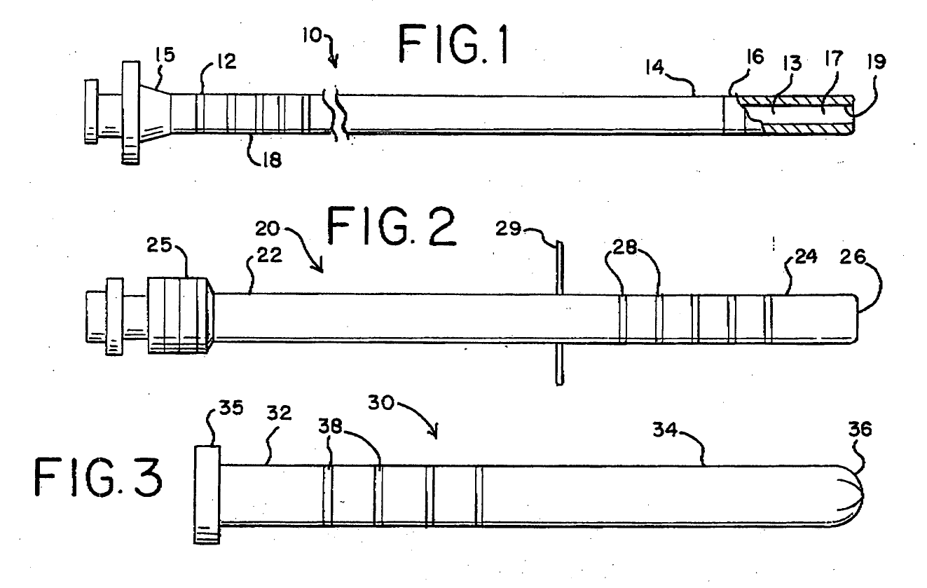

FIGS. 1-3 depict a cellular material transfer catheter system that comprises three catheters, an inner transfer catheter 10, a guide catheter 20, and an outer protective sheath 30. The transfer catheter 10 extends somewhat longer than the guide catheter 20, and the protective outer sheath 30. The inner catheter, transfer catheter 10, includes a passageway 13 of sufficient diameter to hold and deliver cellular material, such as early embryos, gametes (oocyte or sperm), blastocysts, or zygotes that are to be transferred from in vitro culture for in vivo implantation and/or fertilization.

The cellular material or embryo transfer catheter 10 includes a proximal portion 12 and a distal portion 14. The proximal portion may include a hub 15 for interfacing with a syringe for implanting cellular material. The catheter may also include an echogenic tip 16, preferably made of stainless steel, for detecting the distal end via ultrasound. Echogenic tip 16 has an ultrasound reflectivity very different from, and preferably greater than, adjacent portions of embryo transfer catheter 10. The catheter may also have markings 18 at the proximal or distal end indicating a position of the catheter to implantation personnel. The catheter itself is made of relatively soft material, such as polyethylene. Other materials that may be used include urethane, polyolefin, poly-octene-ethylene, polyamides, fluoropolymers including but not limited to polytetrafluoroethylene, and silicone.

The diameter of the passageway and volume of the fluid and material contained therein is preferably minimized to a diameter of no greater than 0.025″, preferably less than 0.023″, and most preferably between 0.018″ and 0.021″. The transfer volume is no greater than 30 μl, more preferably 20 μl, or less, and most preferably between 5 and 15 μl. Clinical experience with this catheter, for IVF/ET having a 0.020″ diameter with a volume of approximately 10 μl, indicates an unexpected increase in pregnancy rates, possibly due to the reduced amount of fluid delivered with the embryos. The reduced transfer volume ostensibly lessens the tendency of embryos to migrate to another section of the uterus, for instance, into the fallopian tubes. By increasing the implantation rate, fewer embryos may be needed, thereby reducing the number of unwanted multiple pregnancies and further risks.

FIG. 2 depicts a guide catheter 20 used coaxially with transfer catheter 10. The guide catheter 20 is of relatively simple construction, and may comprise a proximal portion 22 and a distal portion 24, and distal end 26. The distal end 26 is preferably open rather than closed. The catheter 20 also comprises a hub 25 for interfacing with the embryo transfer catheter 10, and possibly the protective sheath 30. The guide catheter may also comprise markings 28 at the distal portion 24 to guide delivery personnel. The guide catheter also includes cervical stop 29. The guide catheter is preferably somewhat stiffer than the embryo transfer catheter. Materials suitable for the guide catheter are many, so long as the guide catheter is able to hold its shape without drooping or sag during the implantation procedure. Materials used include fluoropolymers such as polytetrafluoroethylene (PTFE), although other materials, as mentioned above, may also be used.

Cervical stop 29 is desirably made from a soft material, such as silicone or urethane. Stop 29 is preferably large enough so that it cannot enter a cervix. A diameter of stop 29 is preferably in the range of about 5-15 mm. The width is preferably about 1-3 mm, sufficiently wide that the stop cannot be bent and deformed easily.

The outer protective sheath 30 of FIG. 3 is also of relatively simple construction. It has a proximal end 32, a distal end portion 34 with a distal end portions 36 and may have a hub or interface 35. Distal portions 36 of the protective sheath pull apart and draw mucus and blood away as the guide catheter emerges from the protective sheath. The sheath may be made with markings 38 for guidance of delivery personnel. The protective sheath is also relatively stiff compared to the embryo transfer catheter. It is important that the guide catheter and the protective sheath be dimensionally stable (do not sag) so that operating personnel may control the exact position of the catheter and the sheath during implantation procedures. The inner transfer catheter should be relatively soft so as to avoid any damage to delicate tissues in the uterus.

The transfer catheter, the guide catheter and the protective sheath catheter are used to implant an embryo into a uterus of a woman. As discussed above, one problem with such implantations is fouling of the distal end of the transfer catheter. The mucousal nature of the cervix, and the presence of mucus and blood, makes the problem an inherent one for any procedure in this area of the body. The present invention solves the problem in the following manner. The three catheters are advanced as a unit through the vagina, through the cervix, and positioned at the internal cervical ostium. The echogenic tip and the markings on one or more of the catheters assist in this operation.

The protective sheath, with the guide catheter still inside the protective sheath, is then retracted to expose the tip of the guide catheter. The distal end of the protective sheath is closed and is impervious to the fouling substances in the cervix, but the protective sheath is also scored or weakened so that the guide catheter is easily advanced through the scored or weakened end portion of the protective sheath. The protective sheath is designed to snap onto the guide catheter when completely retracted. The protective sheath at this point may be covered with mucus or blood or other fouling substances. In practice, these substances cling to the sheath while the guide catheter advances relatively free of the mucus and blood. It is not necessary to retract the protective sheath a great distance; about 1 to 2 cm is sufficient to clear the guide catheter and pull mucus and blood away from the guide catheter tip.

While most of the mucus and blood are retained on the protective sheath, a small amount may cling to the distal (protruding) end of the guide catheter. In practice, this small amount tends to cling to the sides of the guide catheter, rather than the area of the central lumen of the guide catheter. Thus, by extending the guide catheter through the protective sheath, a passageway that is free of fouling substances, such as mucus and blood, is cleared through the central lumen of the guide catheter. All that remains is to advance the transfer catheter through the guide catheter, and to implant the embryo or cellular material. As stated above, the transfer catheter preferably has an echogenic tip to guide operating personnel as to its exact position and to complete the transfer procedure for the embryo or other cellular material. It is preferable to use a syringe and to expel the embryo into the uterus by means of fluid pressure.

Because the delivery catheter is preferably made of a softer (lower durometer) polymer the surface energy density is usually higher, making the embryo more likely to adhere to the inner luminal surface. This is especially critical with a small lumen diameter, since with a typical embryo having a diameter of about 120 micrometers and a blastocyst having a diameter of about 260 micrometers, there is an increased likelihood of problems in delivery. Luminal surface treatments may help reduce friction for the smooth expulsion of oocytes and embryos. Ion beam bombardment is a well-known technique for reducing surface energy density of polymers. Polishing and surface coatings can also offer improvement in friction coefficients for otherwise “sticky” polymers. The luminal surface 19 of the passageway 13 of the distal portion 14 of the delivery catheter is coated with lubricious material 17, such as parylene, to reduce surface energy density. Paralyne coatings may be applied by in-house systems or by vendors, such as Specialty Coating Systems, Indianapolis, Ind., or Parylene Coating Services, Katy Tex., among other vendors. Other coatings, such as PTFE, plasma or corona treatments, may also be used.

In one illustrative embodiment, the protective sheath has an outer diameter of about 6.8 Fr (about 2.27 mm) and has an overall length of about 11 or 16 cm. The guide catheter has an outer diameter of 4.7 Fr (1.57 mm) and an overall length of about 12 or 17 cm. The inner catheter diameter is about 0.483 mm with a length of approximately 19 or 24 cm. The delivery or transfer catheter extends approximately 5 cm beyond the tip of the guiding catheter, and the guide catheter extends about 1 to 2 cm beyond the tip of the protective sheath catheter.

As mentioned above, optional graduated markings 18, 28 can be placed about the proximal portion 12 of the delivery catheter 10 or the distal portion 24 of the guiding catheter 20 to determine the depth of penetration of the guide catheter into the uterus or the amount of delivery catheter 10 to be exposed beyond the distal tip 26 of the guiding catheter 20. Additional graduated markings may also be placed on the guide sheath if desired. The physician or medical professional may use these marks in conjunction with ultrasonic imaging techniques in order to visualize the position of the transfer catheter tip and the patient's uterus.

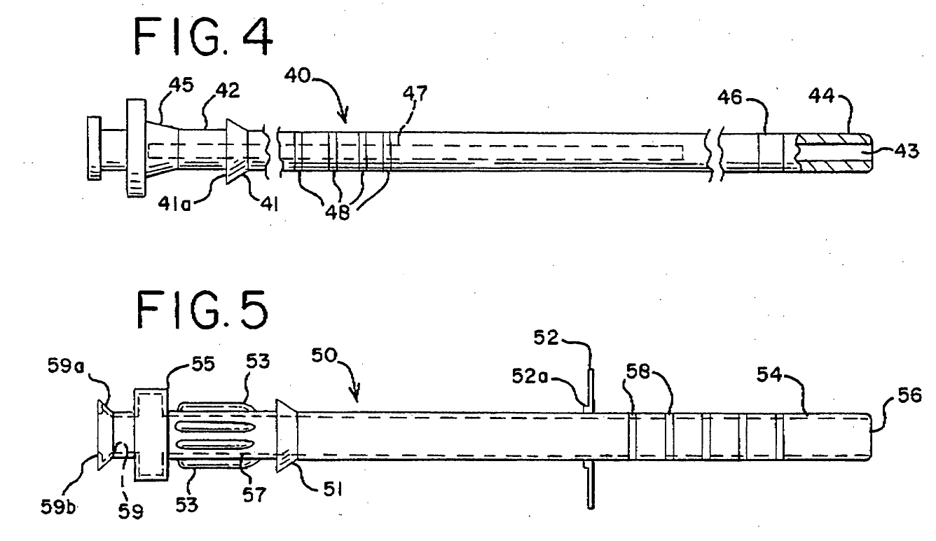

In addition to the delivery catheter embodiment depicted in FIG. 1, the transfer catheter can be made with a stiffened proximal component. FIG. 4 depicts an embryo transfer catheter 40 having a stiffening or reinforcing portion 47 in its proximal portion 42. The embryo transfer catheter 40 also includes a central lumen 43 and a distal portion 44, preferably with an echogenic tip 46. The echogenic tip may be made of stainless steel, or may also take the form of particles embedded into the outer surface of the catheter. It has been found that spherically-shaped metallic particles, or hemispherically-shaped voids or cavities are better for the resulting ultrasonic images. The particles are preferably incorporated into the desired location of the embryo transfer catheter, or possibly into the guide catheter, by molding them into the catheter.

The echogenic tip and the markings on one or more of the catheters assist in this operation. As stated above, the echogenic tip may be made of stainless steel, or may also take the form of particles embedded into the outer surface of the catheter. It has been found that spherically-shaped metallic particles are better for the resulting ultrasonic images. The particles are preferably incorporated into the desired location of the embryo transfer catheter, or possibly into the guide catheter, by molding them into the catheter. If a ring of stainless steel or other suitable material is used, it may be made echogenic by machining or otherwise placing on the surface grooves, bars, lines, bands, dimples, or other patterns which cause reflection, scattering and diffraction of ultrasound or other energy used to guide the surgeon in the placement of the catheter in the uterus.

The proximal portion 42 may also include graduated markings 48 and an interface 45. Reinforcing member 47 may be a stainless steel tube that is bonded to the embryo catheter, preferably by heat or by an adhesive. However, the fit between the reinforcing member and the delivery catheter is typically sufficient that bonding is not required. The reinforcing member may be a cannula on the inside or on the outside of the transfer catheter. An example of a stiffened embryo transfer catheter is polyethylene tubing having a central lumen of 0.019 in (about 0.483 mm) diameter with a 23GXTW stainless steel cannula. An outer cannula, with polyethylene tubing on the inside of the cannula, may also be used.

The transfer catheter and the guide catheter are used to implant an embryo or other cellular material into a uterus of a woman. As discussed above, one problem with such implantations is the proliferation of sizes, especially of guide catheters. In one line of embryo transfer catheters, the lengths of transfer catheter may range from about 18.5 cm to about 23.5 cm, while the guiding catheters may range from 12 cm to 17 cm.

Transfer catheter 40 with proximal portion 42 also includes a male snap on or snap fit feature 41. This feature is a protrusion on an external surface of catheter 40. Snap fit feature 41 has an edge 41a facing the proximal direction, so that edge 41a may interface with a female snap fit or snap on feature on a mating part, such as guide catheter 50 in FIG. 5. Using the snap fit or snap on features, guide catheter 50 may be snap fit over delivery catheter 40. Guide catheter 50 includes a hub 55 at its proximal end, a female snap fit or snap on feature 59, and a relatively soft uterine stop 52 with a short hub 52a. Hub 52a provides a larger interface for stop 52 with catheter 50, holding stop 52 more firmly in place while the catheters are being advanced through the patient's body.

Catheter 50 also has a central lumen 56 and may have marking bands 58 preferably at distal end 54. Catheter 50 has one or more ribs 53 and a reinforcing band 57 which may include connecting hub 55 around the proximal end. The band may be made of any desired, relatively stiffer material suitable for the application, such as PTFE or polyolefin.

Snap on feature 59 includes a space or void 59a for receiving male snap on feature 41 and an edge 59b for mating and interfering with edge 41a of the male snap on feature. The edges form an interference that prevents axial movement of the two components of which the edges are a part in a direction opposed to the direction that caused the engagement. That is, once catheter 40 is placed inside catheter 50, the snap fit features tend to prevent the removal of catheter 40 from catheter 50. Catheter 50 may also have a male snap on feature 51 for assembling a protective sheath to catheter 50. A protective sheath may have a mating female snap on feature to accommodate catheter 50

The catheters described above are preferably used with imaging techniques that allow a doctor or medical professional to visualize the placement of the catheter and the embryo. It is well known that ultrasonic images may be made through the abdomen, i.e., placing an ultrasound transducer on the abdomen of a patient to visualize the internal organs. However, ultrasonic detection using an abdominal technique is usually less than clear, and often not helpful in locating the uterine opening or the precise place in the uterine endometrium at which placement is desired.

It has been discovered that ultrasonic imaging using a vaginal technique may be superior to the abdominal techniques used to date. The physician places an ultrasonic transducer into the vagina, and observes both the uterus and the catheters. As noted above, the transfer catheter desirably has an echogenic tip, or a radiopaque tip, allowing for easier observation with a suitable imaging technique. Using this technique, the physician can then estimate the distance from the cervical os or opening, to the desired location for implantation on the back wall of the uterine endometrium. The physician then calculates the total distance of advance desired into the uterus, and allocates a portion of this distance as the distance between the cervical stop and the distal tip of the guide catheter; the remainder is the distance the distal tip of the transfer catheter is advanced beyond the distal tip of the guide catheter. The physician can then adjust the uterine stop on the guide catheter, limiting the travel of the guide catheter into the uterus. A transfer catheter with marks on its proximal end can assist in this process, since the physician then knows how far the transfer catheter should be advanced and uses these marks, and its echogenic tip, to achieve the desired advance.

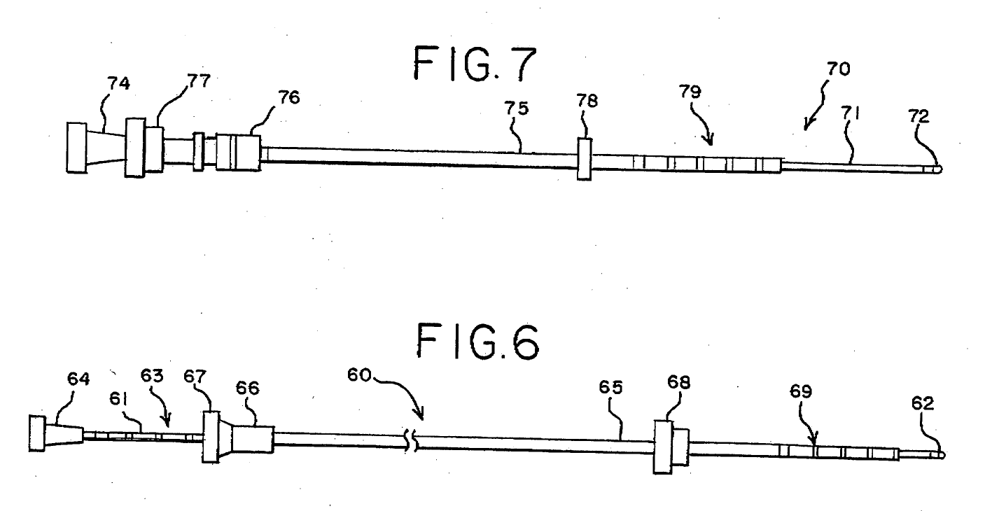

FIGS. 6-7 depict alternative embodiments of the present invention having ultrasonically reflective components or features to enhance visibility under transabdominal or transvaginal ultrasound guidance during embryo transfer. Transfer catheter systems as described above are useful in carrying out this technique. The transfer catheter systems 60, 70 in FIGS. 6-7 may also be used. In FIG. 6, a transfer catheter 61 includes a length of soft plastic or elastomeric tubing, preferably in the range of 80-85 Shore A durometer. The band 62 on the distal tip of transfer catheter 60 is preferably echogenic, but may instead be radiopaque. There are markings, preferably bands 63 spaced at about 1 cm intervals, near the proximal end of transfer catheter 61. Other distances may be used. There is also a fluid connection 64 at the proximal end, such as a female Luer lock adapter (FLLA) for connecting to a source of transfer fluid and embryos. An outer guide catheter 65 is used for guiding the inner transfer catheter to the desired location in the patient. The outer guide catheter also includes a checkflow fitting 66 with a silicone septum 67 for admitting and helping to hold the transfer catheter 61. A cervical positioner or stop 68 may also be placed on outer guide catheter 65 to aid the physician in positioning the catheters. Spaced marks 69 may also be used to help in positioning the catheters. The marks may be placed at any desired interval. 1 cm intervals are presently preferred, but other intervals may be used.

Another transfer catheter system 70 is depicted in FIG. 7. In this embodiment, inner transfer catheter 71 also includes an echogenic tip 72 at its distal end, and a series of spaced markings at its proximal end (not shown). A fluid connector, such as FLLA 74 is attached at the proximal end for accepting medium and embryos to be transferred. Outer guide catheter 75 includes a movable stop or positioner 78 for convenience by the physician. The outer catheter also includes spaced marks 79, preferably at 1 cm intervals. An adapter 76 and a Tuohy-Borst adapter 77 may be placed on guide catheter 75 to lock inner catheter 71 in place when the physician is advancing the catheters together through the patient, or otherwise during the procedure when convenient. The Tuohy-Borst adapter works by compressing the outside of the inner catheter sufficiently that is cannot be moved axially with respect to the outer catheter, and is locked in place.

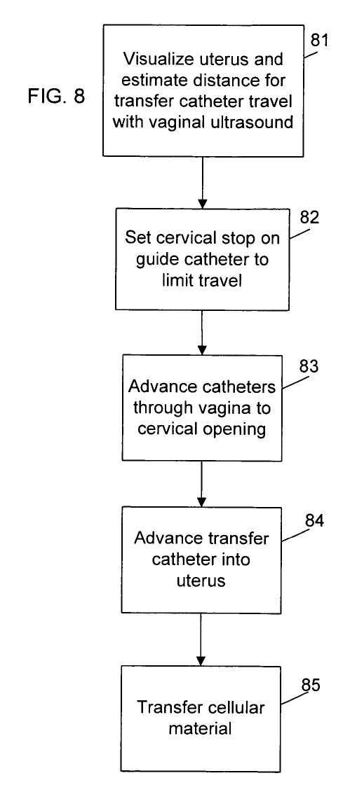

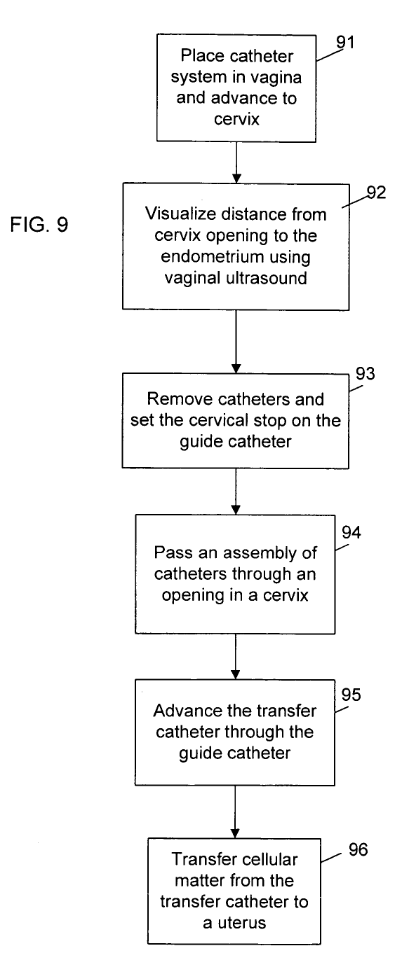

FIGS. 8-9 are flowcharts depicting methods of using a transvaginal ultrasound technique for implantation of an embryo or cellular matter. FIG. 8 depicts a method in which a physician or health-care professional places an ultrasound transducer in the vagina to estimate the distance 81 between the cervical os and the endometrium. This is approximately the total distance the transfer catheter will desirably advance beyond the cervical opening.

The physician will want to advance the guide catheter for a portion of this distance to protect the transfer catheter from mucus and other material that could foul the distal tip of the transfer catheter. The physician then sets the cervical stop 82 on the transfer catheter to limit its travel into the uterus, and then calculates the distance remaining for the transfer catheter to travel. The two catheters, or three if a protective sheath is used, are then locked together using the connectors described above, and the catheters are advanced through the vagina to the cervical opening 83. The physician then unlocks the connectors, allowing the transfer catheter to move axially with respect to the guide catheter, and advances the transfer catheter the remaining desired amount into the uterus 84. The cellular material or embryos may then be transferred into the uterus.

Another technique is described in FIG. 9, in which the catheters are first advanced in order to aid in the step of estimating the distance required for travel into the uterus. A first step is to place the catheter system into the vagina and to advance the system to the cervix 91. This is preferably accomplished using ultrasound imaging. The echogenic tip previously described may assist in visualizing the distance from the cervical opening to the desired area, the endometrium, by using an ultrasonic transducer placed into the vagina 92. Once the distance is estimated, the catheters may be removed and the cervical stop set in place on the guide catheter using the markings on the distal end of the guide catheter. The physician also calculates the distance the transfer catheter will have to be advanced for the most desirable implantation.

The catheters are then connected or locked together, and passed through the vagina and through the cervical opening 94. The transfer catheter is advanced through the guide catheter 95 by the desired amount, preferably using the spaced marks on the proximal end of the transfer catheter. The embryos or cellular material is then transferred 96 from the transfer catheter to the uterus. Other techniques of imaging, estimating, and advancing may be used.

Embodiments of the present invention may be made from one or more of the materials listed above, and may be used for any of the procedures described herein. While the cervical stop is preferably made from a soft urethane or silicone material, it may be made from any other medically-acceptable elastomer, such as nitrile, or from a medically-acceptable plastic, such as polyethylene or polypropylene. The catheters may also be made from alternative materials, although the transfer catheter is preferably made from a very soft plastic or elastomer, in a Shore A hardness from about 80-85. Thermoplastic olefin elastomers may be useful applied to transfer catheter applications. Several grades of polyolefin from DuPont Dow Elastomers may be useful for this purpose, including olefins made from blends of ethylene and octene. The soft grades are preferred, such as Engage® 8100 and 8480, especially preferred is 8003, which has a slightly higher density than 8100 and 8480.

It is possible to obtain a number of advantages of the present invention by using methods other than those specifically described above. For instance, one may place a transfer catheter or other device with an echogenic feature near the cervical os, and observe via abdominal ultrasound or fluoroscopy the distance from the cervical opening to the desired location for implantation within the uterus. Implantation may then be accomplished using the catheter system and cervical stop described above. While it is believed that this method is inferior to one using vaginal ultrasound, the use of the cervical stop would be an improvement over present methods that do not use a cervical stop.

It is intended that the foregoing detailed description be regarded as illustrative rather than limiting, and that it be understood that it is the following claims, including all equivalents, that are intended to define the spirit and scope of this invention.

What is claimed is:

1. A transfer system for cellular material, comprising:
a transfer catheter comprising a distal end detectably different from adjacent portions of the catheter;
a guide catheter for holding and guiding the transfer catheter, the guide catheter further comprising a movable cervical stop; and
a locking mechanism for fixing the position of the transfer catheter with respect to the guide catheter, wherein the transfer system is suitable for guidance using a transvaginal ultrasound technique.

2. The system of claim 1, further comprising a plurality of evenly-spaced marks on an outer surface of the guide catheter.

3. The system of claim 1, further comprising a plurality of evenly-spaced marks on an outer surface of the transfer catheter.

4. The system of claim 1, wherein the cervical stop comprises a soft elastomeric or plastic disc.

5. The system of claim 1, the detectable difference of the distal end comprises an echogenic or radiopaque band.

6. The system of claim 1, wherein the transfer catheter further comprises at least one material selected from the group consisting of polyethylene, urethane, polyolefin, polyethylene terephthlate, polyamide, silicone, and polytetrafluoroethylene.

7. The system of claim 1, wherein the transfer catheter has an inner diameter from about 0.018 inches (0.45 mm) to about 0.025 inches (0.63 mm).

8. The system of claim 1, wherein the transfer catheter has a transfer volume of from about 5 μl to about 15 μl.

9. The system of claim 1, wherein the transfer catheter further comprises a snap-fit feature.

10. The system of claim 1, wherein the locking mechanism comprises a compression fitting between the guide catheter and the transfer catheter.

11. A catheter transfer system, comprising:
a soft inner transfer catheter comprising a connector for fluid transfer on a proximal end and an echogenic or radiopaque marker on a distal end;
an outer guide catheter comprising a cervical stop and a series of spaced marks on a distal end of the outer guide catheter; and
a locking mechanism for fixing a position of the inner and outer catheters with respect to one another.

12. The system of claim 11, further comprising a series of spaced marks on a proximal end of the transfer catheter.

13. The system of claim 11, wherein the locking mechanism comprises a compression fitting.

14. The system of claim 11, further comprising an outer protective sheath.

15. A method of transferring an embryo, the method comprising:
adjusting a cervical stop on a catheter transfer system;
placing the catheter transfer system near an opening of a cervix;
observing at least one echogenic or radiopaque feature of the catheter transfer system using an ultrasound transducer placed near a vagina and optionally observing at least one echogenic or radiopaque feature of the catheter transfer system using an ultrasound transducer placed near an abdomen;
advancing a transfer catheter to a desired position in a uterus; and
transferring the embryo into the uterus.

16. The method of claim 15, wherein adjusting takes place by moving the cervical stop among a series of marked intervals on an external surface of a guide catheter.

17. The method of claim 15, wherein the catheter transfer system comprises a transfer catheter and a guide catheter, and further comprising fixing a position of the transfer catheter with respect to the guide catheter before the step of advancing.

18. A method of transferring cellular material, the method comprising:
placing at least one catheter near an opening of a cervix;
observing a position of the at least one catheter using an ultrasound transducer placed in or near a vagina and optionally using an ultrasound transducer placed near an abdomen;
adjusting a cervical stop on the at least one catheter;
advancing the at least one catheter into the cervix and uterus until the stop is reached;
observing the position of a distal end of the at least one catheter using at least one ultrasound transducer; and
transferring the cellular material.

19. The method of claim 18, wherein the steps of observing use an echogenic or radiopaque band on a distal end of the at least one catheter.

20. The method of claim 18, wherein the step of adjusting or the step of advancing uses a plurality of marked intervals on a guide catheter or a transfer catheter.
